(19) United States
(12) Patent Application Publication
(10) Pub. No.: US 2016/0067426 A1
(43) Pub. Date: Mar. 10, 2016

(54) Lighted Syringe Shrouding Device

(71) Applicant: Shweta A. Ujaoney, Richmond, VA (US)

(72) Inventor: Shweta A. Ujaoney, Richmond, VA (US)

(21) Appl. No.: 14/482,013

(22) Filed: Sep. 10, 2014

(51) Int. Cl. A61M 5/42; A61M 5/31

(52) U.S. Cl. CPC A61M 5/427; A61M 5/3129; A61M 2205/59; A61M 2205/583; A61M 2005/3142

(57) ABSTRACT

A lighted syringe shrouding device lights an injection site while shrouding a syringe from view to reduce patient anxiety. The device includes an elongated tube configured to receive a syringe such that a needle of the syringe is extendable out of a first end of the tube and a plunger of the syringe is accessible through a second end. A rib extending from an interior surface of the tube engages the syringe allows the needle of the syringe to be positioned fully within the tube until ready for being fully extended from the tube. A light is coupled to the tube to illuminate an injection site adjacent to the first end of the tube.

### BACKGROUND OF THE DISCLOSURE

### Field of the Disclosure

The disclosure relates to shrouding devices and more particularly pertains to a new shrouding device for lighting an injection site while shrouding a syringe from view to reduce patient anxiety.

### SUMMARY OF THE DISCLOSURE

An embodiment of the disclosure meets the needs presented above by generally comprising an elongated tube configured to receive a syringe such that a needle of the syringe is extendable out of a first end of the tube and a plunger of the syringe is accessible through a second end. A rib extending from an interior surface of the tube engages the syringe allows the needle of the syringe to be positioned fully within the tube until ready for being fully extended from the tube. A light is coupled to the tube to illuminate an injection site adjacent to the first end of the tube.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of a lighted syringe shrouding device according to an embodiment of the disclosure.

FIG. 2 is a bottom rear side perspective view of an embodiment of the disclosure.

FIG. 3 is a side view of an embodiment of the disclosure.

FIG. 4 is a rear view of an embodiment of the disclosure.

FIG. 5 is a cross-sectional view of an embodiment of the disclosure in use taken along line 5-5 of FIG. 4.

FIG. 6 is a cross-sectional view of an embodiment of the disclosure similar to FIG. 5 showing a syringe in a fully extended in use position.

### DESCRIPTION OF THE PREFERRED EMBODIMENT

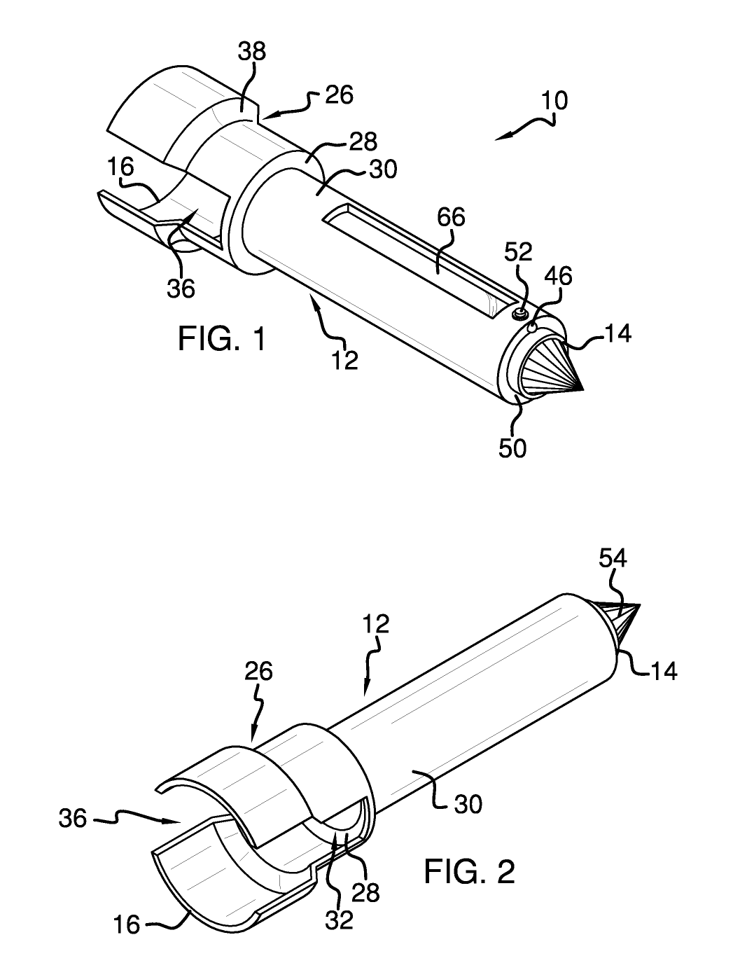

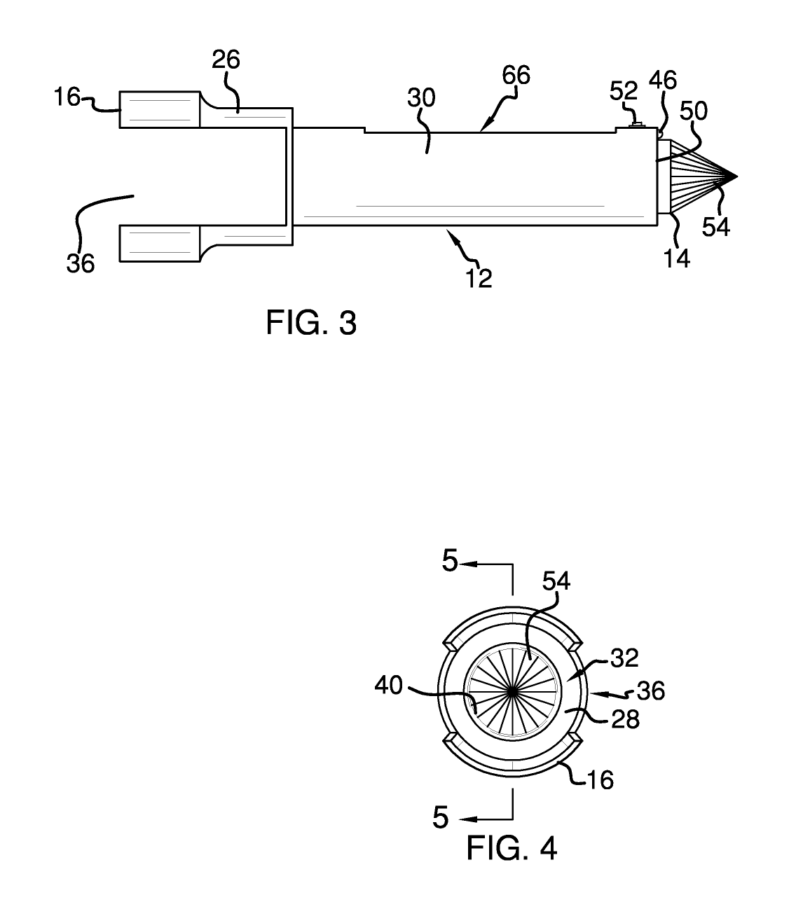

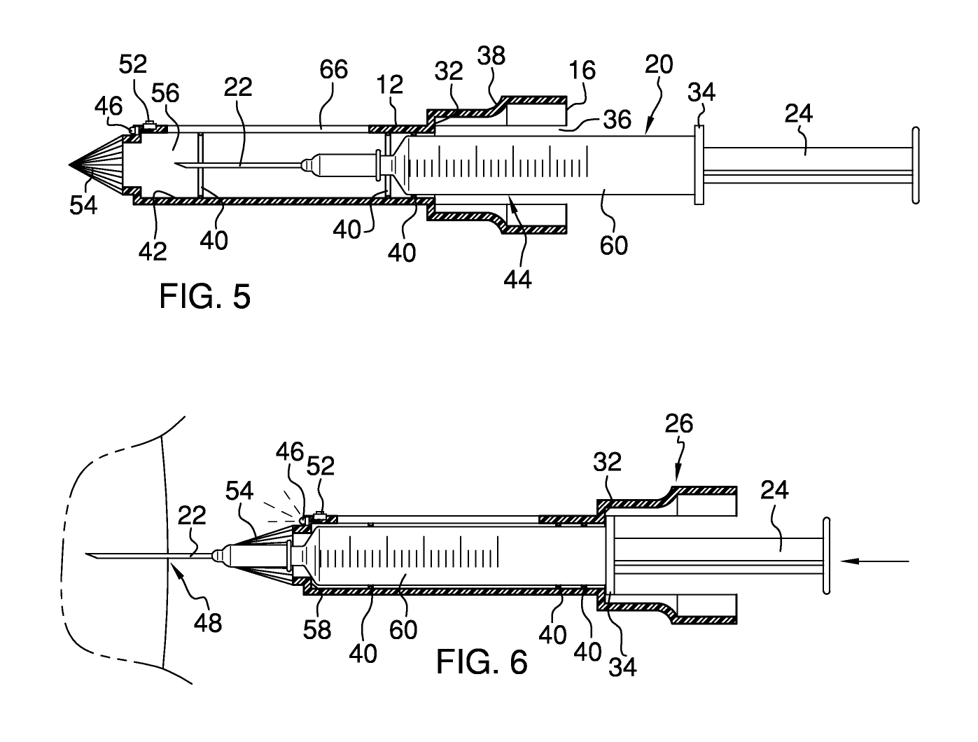

With reference now to the drawings, and in particular to FIGS. 1 through 6 thereof, a new shrouding device embodying the principles and concepts of an embodiment of the disclosure and generally designated by the reference numeral 10 will be described.

As best illustrated in FIGS. 1 through 6, the lighted syringe shrouding device 10 generally comprises a tube 12 which is elongated. The tube 12 has a first end 14 and a second end 16. The first end 14 is open and the second end 16 is open. Thus, the tube 12 is configured to receive a syringe 20 such that a needle 22 of the syringe 20 is extendable out of the first end 14 and a plunger 24 of the syringe 20 is accessible extending out of or through the second end 16. The tube 12 further has a flared section 26 adjacent the second end 16 wherein the second end 16 is configured for facilitating manipulation of the plunger 24 of the syringe 20 when the syringe 20 is fully inserted into the tube 12. The flared section 26 has a transverse section 28 relative to a main section 30 of the tube 12. The transverse section 28 defines a stop 32 configured to engage a flange 34 of the syringe 20 adjacent to the plunger 24 of the syringe 20 defining a fully inserted position for the syringe 20 in the tube 12. The flared section 26 is also structured to include a slot 36 extending transversely across the flared section 26 wherein the slot 36 is configured to facilitate manipulation of the plunger 24 of the syringe 20. The flared section 26 comprises a circumferential arcuate portion 38 flaring outwardly relative to the main section 30 of the tube 12 wherein the flared section 26 is configured for being engaged by fingers to facilitate pushing of the plunger 24 of the syringe 20 towards the first end 14 of the tube 12. The tube 12 may also include a window 66 allowing viewing of the syringe 20 within the tube 12. The tube 12 may further provide a decorative or ornamental outward appearance further disguising the nature of the device 10. The outward appearance of the tube 12 may correspond to a variety of designs which may be found attractive or appealing to a child.

At least one rib 40 extends from an interior surface 42 of the tube 12 such that the rib 40 is configured to engage the syringe 20 wherein the syringe 20 is stably positioned in the tube 12 in a partially inserted position 44. In the partially inserted position the needle 22 of the syringe 20 is positioned fully within the tube 12. The rib 40 may be one of a plurality of ribs 40 positioned in spaced relationship along a longitudinal length of the tube 12.

A light 46 is coupled to the tube 12. The light 46 provides illumination directed outwardly relative to the first end 14 of the tube 12 wherein the light 46 is configured to illuminate an injection site 48 adjacent to the first end 14 of the tube 12. The light 46 is positioned on a circumferential surface 50 facing the first end 14 of the tube 12. The light may be a light emitting diode or the like. A switch 52 is coupled to the tube 12. The switch 52 is electrically coupled to the light 46 wherein the light 46 is selectively illuminated by manipulation of the switch 52. The switch 52 is positioned adjacent to the light 46 and may be a button pressed to operate the switch 52.

A plurality of bristles 54 is coupled to the first end 14 of the tube 12. The bristles 54 are arranged to taper extending away from the first end 14 of the tube 12. The bristles 54 are bunched together wherein the bristles 54 obscure viewing of an interior 56 of the tube 12 through the first end 14 of the tube 12. Thus, the bristles 54 prevent viewing of the needle 22 of the syringe while the syringe 20 is in the partially inserted position 44.

A lip 58 is positioned in the tube 12. The lip 58 is positioned adjacent the first end 14 of the tube 12 wherein the lip 58 is configured for preventing a barrel 60 of the syringe 20 from passing through the first end 14 of the tube 12. The device 10 may be sized corresponding to the shape of the syringe 20 such that the lip 58 is engaged by the barrel 60 substantially simultaneously to the flange 34 abutting the stop 32.

In use, the device 10 prevents a patient such as a child from viewing the needle 22 of the syringe 20. Thus, the device 10 may be used for medical and dental applications. The light 46 is used to illuminate the injection site 48 as may be needed particularly in dentistry where the mouth is dark and may be small when the patient is a child. Further, the bristles 54 may be soft and used to apply a topical anesthetic prior to fully inserting the syringe 20 into the tube 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A lighted syringe shrouding device comprising:
a tube, said tube being elongated, said tube having a first end and a second end, said first end being open, said second end being open wherein said tube is configured to receive a syringe such that a needle of the syringe is extendable out of said first end and a plunger of the syringe is accessible through said second end;
a rib extending from an interior surface of said tube wherein said rib is configured to engage the syringe wherein the syringe is positionable in the tube such that the needle of the syringe is positioned fully within said tube;
a light coupled to said tube, said light providing illumination directed outwardly relative to said first end of said tube wherein said light is configured to illuminate an injection site adjacent to said first end of said tube; and
a plurality of bristles coupled to said first end of said tube, said bristles being arranged to taper extending away from said first end of said tube, said bristles being bunched together and covering said first end of said tube wherein said bristles are configured for obscuring viewing of an interior of said tube through said first end of said tube.

2. (canceled)

3. The device of claim 1, further comprising said rib being one of a plurality of said ribs, said ribs being positioned in spaced relationship along a longitudinal length of said tube.

4. The device of claim 1, further comprising said tube having a flared section adjacent said second end wherein said second end is configured for facilitating manipulation of the plunger of the syringe when the syringe is fully inserted into said tube.

5. The device of claim 1, further comprising a lip positioned in said tube, said lip being positioned adjacent said first end of said tube wherein said lip is configured for preventing a barrel of the syringe from passing through said first end of said tube.

6. The device of claim 4, further comprising said flared section having a transverse section relative to a main section of said tube, said transverse section defining a stop configured to engage a flange of the syringe adjacent to the plunger of the syringe defining a fully inserted position for the syringe in said tube.

7. The device of claim 4, further comprising said flared section being structured to include a slot extending transversely across said flared section wherein said slot is configured to facilitate manipulation of the plunger of the syringe.

8. The device of claim 4, further comprising said flared section comprising a circumferential arcuate portion flaring outwardly relative to a main section of said tube wherein said flared section is configured for being engaged by fingers to facilitate pushing of the plunger of the syringe towards said first end of said tube.

9. The device of claim 1, further comprising a switch coupled to said tube, said switch being electrically coupled to said light wherein said light is selectively illuminated by manipulation of said switch.

10. The device of claim 1, further comprising said light being positioned on a circumferential surface facing said first end of said tube.

11. The device of claim 9, further comprising said switch being positioned adjacent to said light.

12. A lighted syringe shrouding device comprising:
a tube, said tube being elongated, said tube having a first end and a second end, said first end being open, said second end being open wherein said tube is configured to receive a syringe such that a needle of the syringe is extendable out of said first end and a plunger of the syringe is accessible through said second end, said tube having a flared section adjacent said second end wherein said second end is configured for facilitating manipulation of the plunger of the syringe when the syringe is fully inserted into said tube, said flared section having a transverse section relative to a main section of said tube, said transverse section defining a stop configured to engage a flange of the syringe adjacent to the plunger of the syringe defining a fully inserted position for the syringe in said tube, said flared section being structured to include a slot extending transversely across said flared section wherein said slot is configured to facilitate manipulation of the plunger of the syringe, said flared section comprising a circumferential arcuate portion flaring outwardly relative to a main section of said tube wherein said flared section is configured for being engaged by fingers to facilitate pushing of the plunger of the syringe towards said first end of said tube;
a rib extending from an interior surface of said tube wherein said rib is configured to engage the syringe wherein the syringe is positionable in the tube such that the needle of the syringe is positioned fully within said tube, said rib being one of a plurality of said ribs, said ribs being positioned in spaced relationship along a longitudinal length of said tube;
a light coupled to said tube, said light providing illumination directed outwardly relative to said first end of said tube wherein said light is configured to illuminate an injection site adjacent to said first end of said tube, said light being positioned on a circumferential surface facing said first end of said tube;
a switch coupled to said tube, said switch being electrically coupled to said light wherein said light is selectively illuminated by manipulation of said switch, said switch being positioned adjacent to said light;
a plurality of bristles coupled to said first end of said tube, said bristles being arranged to taper extending away from said first end of said tube, said bristles being bunched together and covering said first end of said tube wherein said bristles are configured for obscuring viewing of an interior of said tube through said first end of said tube; and
a lip positioned in said tube, said lip being positioned adjacent said first end of said tube wherein said lip is configured for preventing a barrel of the syringe from passing through said first end of said tube.
